(19) United States
(12) Patent Application Publication
(10) Pub. No.: US 2026/0069312 A1
(43) Pub. Date: Mar. 12, 2026

(54) NEEDLE DELIVERED ELECTRODE SYSTEM

(71) Applicant: CVRX, INC., Minneapolis, MN (US)

(72) Inventors: Paul Pignato, Jacksonville, FL (US); Gregory Sundberg, Stillwater, MN (US); Luke Parker, Cottage Grove, MN (US); Seth Wilks, Valparaiso, IN (US)

(21) Appl. No.: 19/125,974

(22) PCT Filed: Nov. 16, 2023
(86) PCT No.: PCT/US2023/080127

§ 371 Date: Apr. 30, 2025

Related U.S. Application Data
(60) Provisional application No. 63/426,017, filed on Nov. 16, 2022.
(60) Provisional application No. 63/426,021, filed on Nov. 16, 2022.

(51) Int. Cl. A61B 17/34; A61B 17/00

(52) U.S. Cl. CPC A61B 17/3468; A61B 17/3494; A61B 2017/00867

(57) ABSTRACT

Systems and methods for implanting a baroreflex activation device are disclosed. A baroreflex activation system can include a plunger, a needle having a body, a lumen, a distal tip having an aperture and a proximal end opposite the distal tip, and a baroreflex activation device having a distal electrode and one or more proximal electrodes. The distal electrode can be connected to the one or more proximal electrodes via a link housing one or more conductors extending from the needle to the distal electrode. The distal electrode can include an anchoring feature configured to connect the baroreflex activation device to a carotid. The baroreflex activation device can include one or more shape elements configured to bias the link into an arcuate shape, such that the distal electrode contacts a first portion of the carotid, and the one or more proximal electrodes contact a second, adjacent portion of the carotid.

### CROSS-REFERENCE TO RELATED APPLICATION

This application claims the benefit of U.S. Provisional Ser. No. 63/426,017, filed Nov. 16, 2022, and the benefit of U.S. Provisional Ser. No. 63/426,021, filed Nov. 16, 2022, both of which are incorporated by reference herein in their entireties.

### TECHNICAL FIELD

The present disclosure relates generally to surgical implant systems, methods, and devices. More particularly, the present disclosure relates to improved systems and methods for implanting a baroreflex activation therapy device.

### BACKGROUND

One approach for treating hypertension, heart failure, or other cardiovascular disease states is baroreflex activation therapy (or “BAT”), which comprises stimulation of baroreceptors and/or associated nerves or nerve structures of a patient. Baroreceptors are sensory nerve ends that are profusely distributed within the walls of the major arteries, as well in the heart, aortic arch, carotid sinus or arteries, and in the low-pressure side of the vasculature such as the pulmonary artery and vena cava. Baroreceptor signals are used to activate a number of body systems which collectively may be referred to as the baroreflex system. Baroreceptors are connected to the brain via the nervous system. Thus, the brain is able to detect changes in blood pressure, which can be related to, or indicative of, cardiac output.

Early approaches to BAT systems and associated implant procedures typically required relatively large incisions on one or both sides of a patient's neck to create sufficient access to the target vasculature in the area of the carotid sinus. The carotid artery was dissected free, one or more electrode pads were wrapped around the artery and sutured in place.

Current generation implantable BAT devices and systems offer pulse generator housings and associated therapy electrodes with reduced form factors as compared to early systems. One such system, described in U.S. Pat. No. 8,437,867 to Murney et al., includes an implantable pulse generator and associated circuitry contained within a hermetically sealed housing, an elongate flexible electrical lead connectable to the housing, and a monopolar electrode structure coupled with the electrical lead.

Current generation implantable BAT devices and systems also offer improvements to the implant procedure. Smaller electrode structures typically allow smaller incisions on the patient. The electrode structure described in the above-mentioned U.S. Pat. No. 8,437,867 can be implanted via a minimally invasive approach, as described therein.

A recent improvement to the implant procedure for BAT devices and systems involves percutaneous, minimally invasive methods and related devices, as described in published PCT application WO 2020/037145 to Pignato et al. The percutaneous implant methods and devices described therein represent an improvement over prior approaches, however, opportunities exist for further refinements to electrode design, electrode fixation, and delivery tools and techniques. The present disclosure addresses these concerns.

### SUMMARY

The following are some objectives of the present disclosure:
Provide improved systems and methods for implanting a baroreflex activation device into a patient;
Enable fixation of the baroreflex activation device to the carotid artery using a combination of active and passive fixation elements;
Eliminate the need to use active fixation elements to fixate the baroreflex activation device onto the carotid artery, relying instead on one or more passive elements embedded within the baroreflex activation device; and
Enable a baroreflex activation therapy system to incorporate a monopolar, bipolar, or multipolar baroreflex activation device during implantation into a patient.

In embodiments of the present disclosure, a method for implanting a baroreflex activation device into a patient may include inserting a needle into the patient at an implant site having a carotid artery, the needle having a body and a lumen defined along the body, the needle inserted in a first direction toward the carotid artery. The method may further include loading the baroreflex activation device into a proximal end of the needle, the baroreflex device having a distal electrode connected to one or more proximal electrodes via a link. The method may further include advancing the baroreflex activation device along the lumen until the distal electrode of the baroreflex activation device extends beyond a distal tip of the needle.

The method may further include positioning the distal electrode and the one or more proximal electrodes of the baroreflex activation device against the carotid artery, wherein the link of the baroreflex activation device is biased into an arcuate shape using a shape element, such that the distal electrode contacts a first portion of the carotid artery and the one or more proximal electrodes contact a second, adjacent portion of the carotid artery. The method may further include retracting the needle in a second direction opposite the first direction until the needle is removed from the patient.

In embodiments of the present disclosure, a baroreflex activation system may include a plunger, a needle having a body, a lumen defined along the body, a distal tip having an aperture, and a proximal end opposite the distal tip. The baroreflex activation system may further include a baroreflex activation device having a distal electrode and one or more proximal electrodes. The distal electrode may be connected to the one or more proximal electrodes via a link, the link housing one or more conductors extending from the needle to the distal electrode. The one or more conductors may electrically couple the distal electrode to the one or more proximal electrodes. The distal electrode may include an anchoring feature configured to connect the baroreflex activation device to a carotid artery. The baroreflex activation device may include one or more shape elements configured to bias the link into an arcuate shape, such that the distal electrode contacts a first portion of the carotid artery, and the one or more proximal electrodes contact a second, adjacent portion of the carotid artery.

In an embodiment, an improved introducer needle is provided. The needle may include a hub portion, a body portion defining a lumen therein, a closed tip, an exit port, and a diverter. In embodiments, the exit port is on a side or surface of the needle body, proximal of the closed tip. In embodiments, the needle may be echogenic to allow visualization of the exit port during an implant procedure. A diverter is arranged at the end of the lumen, and may be configured as a ramp, wedge, slope or curve, among other examples. The diverter and exit port are configured such that a guidewire advanced through the lumen will, upon contacting the diverter, be directed or urged at an angle out of the exit port. Such an arrangement provides an exit for the guidewire which is off-axis from the lumen.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of a current generation baroreflex activation therapy system, according to embodiments of the disclosure.

FIG. 2 is a perspective view of an introducer needle, according to embodiments of the disclosure.

FIG. 3 is a perspective view of an introducer needle, according to embodiments of the disclosure.

FIG. 4A is a perspective view of a needle delivered electrode system with a monopolar baroreflex activation device, according to embodiments of the disclosure.

FIG. 4B is a perspective view of a needle delivered electrode system with a bipolar baroreflex activation device, according to embodiments of the disclosure.

FIG. 4C are cross-sectional views of a baroreflex activation device with shape elements, according to embodiments of the disclosure.

FIG. 4D is a perspective view of a needle delivered electrode system with a bipolar baroreflex activation device, according to embodiments of the disclosure.

FIG. 4E is a perspective view of a needle delivered electrode system with a bipolar baroreflex activation device, according to embodiments of the disclosure.

FIG. 4F is a perspective view of a needle delivered electrode system with a multipolar baroreflex activation device, according to embodiments of the disclosure.

FIG. 5A is a perspective view of a needle delivered electrode system in an unextended position, according to embodiments of the disclosure.

FIG. 5B is a perspective view of a needle delivered electrode system in a partially extended position, according to embodiments of the disclosure.

FIG. 5C is a perspective view of a needle delivered electrode system in an extended position, according to embodiments of the disclosure.

FIG. 6A is a cross-sectional view of a needle delivered electrode system, according to embodiments of the disclosure.

FIG. 6B is a cross-sectional detail view of the needle delivered electrode system shown in FIG. 6A, according to embodiments of the disclosure.

FIG. 7A is a perspective view of a needle delivered electrode system with an anchoring feature, according to embodiments of the disclosure.

FIG. 7B is a perspective view of the needle delivered electrode system with anchoring feature shown in FIG. 7A, according to embodiments of the disclosure.

FIG. 8A is a perspective view of a needle delivered electrode system with an anchoring feature in an undeployed position, according to embodiments of the disclosure.

FIG. 8B is a perspective view of the needle delivered electrode system with anchoring feature shown in FIG. 8A, in a partially deployed position, according to embodiments of the disclosure.

FIG. 8C is a perspective view of the needle delivered electrode system with anchoring feature shown in FIGS. 8A and 8B, according to embodiments of the disclosure.

FIG. 9A is a perspective view of a needle delivered electrode system with an anchoring feature deployed around a carotid sinus, according to embodiments of the disclosure.

FIG. 9B is a perspective view of a needle delivered electrode system with an anchoring feature deployed around a carotid sinus, according to embodiments of the disclosure.

FIG. 9C is a perspective view of a needle delivered electrode system with an anchoring feature deployed around a carotid sinus, according to embodiments of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the disclosure.

For information pertaining to the cardiovascular, circulatory and nervous systems, as well as baroreceptor and baroreflex therapy systems that may be used in whole or in part with embodiments of the present disclosure, reference is made to the following commonly assigned published applications and patents: U.S. Published Pat. Application Nos. 2006/0004417 to Rossing et al., 2006/0074453 to Kieval et al., and 2008/0082137 to Kieval et al.; U.S. Pat. No. 6,522,926 to Kieval et al., U.S. Pat. No. 6,850,801 to Kieval et al., U.S. Pat. No. 6,985,774 to Kieval et al., U.S. Pat. No. 7,480,532 to Kieval et al., U.S. Pat. No. 7,499,747 to Kieval et al., U.S. Pat. No. 7,835,797 to Rossing et al., U.S. Pat. No. 7,840,271 to Kieval et al., U.S. Pat. No. 8,086,314 to Kieval, U.S. Pat. No. 8,326,430 to Georgakopoulos et al., and U.S. Pat. No. 9,345,877 to Pignato et al., the disclosures of which are hereby incorporated by reference in their entireties.

Embodiments of the present disclosure generally pertain to improved devices and methods for implanting a baroreflex activation therapy system. In embodiments, at least some of the components of the baroreflex activation therapy system can be as described in U.S. Pat. No. 8,437,867 to Murney et al., the disclosure of which is incorporated by reference herein in its entirety. A description of percutaneous, minimally invasive methods and related devices is included in published PCT application WO 2020/037145 (“the '145 PCT publication”) to Pignato et al., the disclosure of which is hereby incorporated by reference in its entirety.

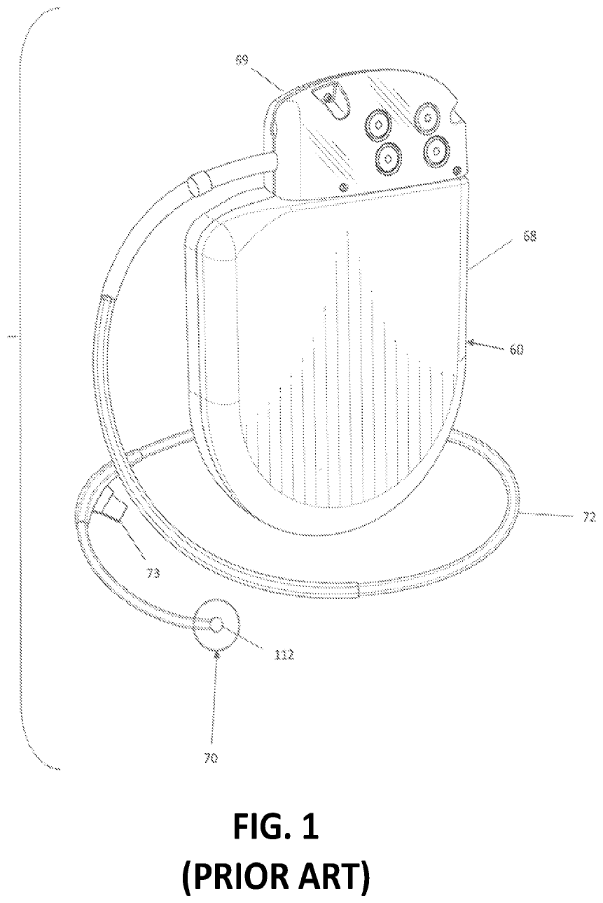

Referring now to FIG. 1, an embodiment of a current generation baroreflex activation therapy system 90 is depicted, including a control system 60, a baroreflex activation device 70, and a lead 72. In embodiments, control system 60 may alternately be referred to as an implantable medical device, an implantable pulse generator, or the like. Control system 60 is understood to include the necessary circuitry and associated components for generating and delivering electrical therapy signals, and other functions. Control system 60 is configured to be implanted within a patient and includes a hermetically sealed housing 68, a header 69 adapted to facilitate connection of one or more leads 72, as well as a case electrode 67 on at least a portion of the outer surface of housing 68. Electrode 67 may also be referred to as an indifferent electrode, common electrode, or reference electrode. As used herein, the words “housing,” “enclosure,” “case,” and “can” are synonymous when used to refer to housing 68.

The control system 60 is configured to generate a control signal (also referred to as a therapy signal), which activates, deactivates or otherwise modulates the baroreflex activation device 70. In an embodiment, the therapy signal is in the range of about 1 to 10 volts, at a rate between 5 Hz and 200 Hz, with an amplitude between 1 and 50 milliamps. Typically, activation of the baroreflex activation device 70 via the control signal results in activation of baroreceptors of a patient. Alternatively, deactivation or modulation of the baroreflex activation device 70 may cause or modify activation of the baroreceptors. The baroreflex activation device 70 may include a wide variety of devices which utilize electrical means, such as electrodes, to activate baroreceptors.

The memory of the control system 60 may contain data related to the therapy signal, a sensor signal, and/or values and commands provided by an input device, such as an external programmer operated by a patient or a clinician. The memory may also include software containing one or more algorithms defining one or more functions or relationships between the therapy signal and the sensor signal. The algorithm may dictate activation or deactivation therapy signals depending on the sensor signal or a mathematical derivative thereof. The algorithm may dictate an activation or deactivation therapy signal when the sensor signal falls below a lower predetermined threshold value, rises above an upper predetermined threshold value or when the sensor signal indicates a specific physiologic event. The memory may also include software containing one or more algorithms for determining patient physiological parameters based on a measured parameter.

In embodiments, baroreflex activation device 70 may comprise an electrode structure 78 which generally includes an electrode 112 mounted on, integrated with, or otherwise coupled to a backer 114. In an embodiment, electrode structure 78 may be considered to be part of lead 72. Electrode 112 may comprise platinum iridium, and may include a surface treatment, such as iridium oxide or titanium nitride and/or can include steroid, anti-inflammatory, antibiotic and/or analgesic compounds, for example. In an example embodiment, electrode 112 may have a diameter of about 1 millimeter, and backer 114 may have a diameter of about 6 millimeters. In an embodiment, electrode 112 comprises a cathode, while reference electrode 67 or housing 68 of control system 60 may comprise an anode.

As described in the '145 PCT publication referenced previously, an implant system for implanting an electrode structure and lead in a minimally invasive procedure may include a guidewire delivery tool used with an introducer needle. The guidewire may be pre-curved such that when the guidewire is advanced out of the needle inserted in a neck of the patient, the tip of the guidewire will advance toward the underside of the skin of the patient and then pushed through the skin, creating a pathway for subsequent lead delivery and electrode fixation. However, during some procedures, proper introduction and placement of the guidewire using the techniques described in the '145 PCT publication can prove challenging.

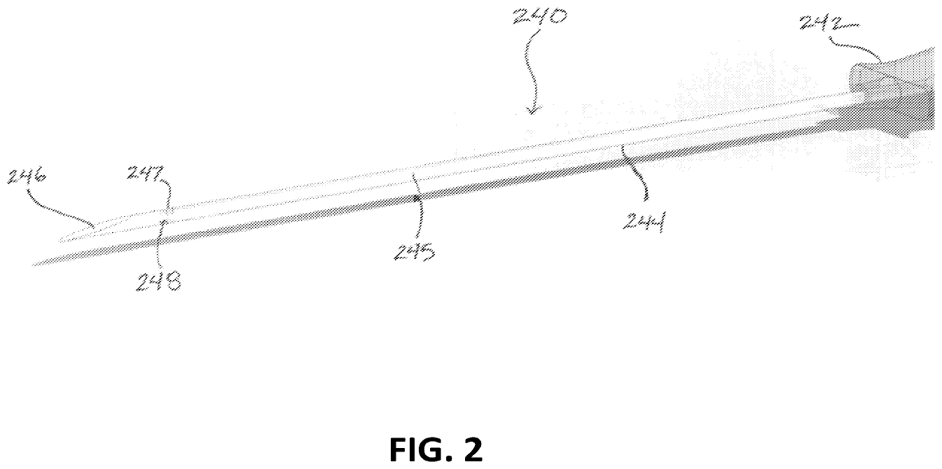

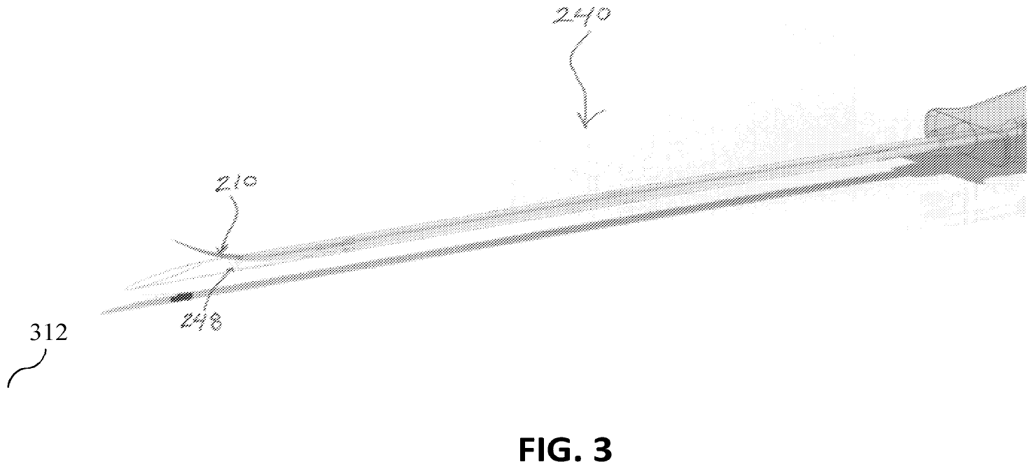

Referring now to FIGS. 2 and 3, an improved introducer needle 240 is depicted. Needle 240 may include a hub portion 242, a body portion 244 defining a lumen 245 therein, a closed tip 246, an exit port 247, and a diverter 248. In contrast to prior introducer needles which feature an open distal tip for passage of a guidewire therethrough, embodiments of the present disclosure include an exit port 247 on a side (or surface) of the needle body 244, proximal of the closed tip 246. In embodiments, needle 240 may be echogenic to allow visualization of exit port 247 during an implant procedure.

Diverter 248 is arranged at the end of lumen 245, and may be configured as a ramp, wedge, slope or curve, among other examples. Diverter 248 and exit port 247 are configured such that a guidewire 210 advanced through lumen 245 will, upon contacting diverter 248, be directed or urged at an angle out of exit port 247, as depicted in FIG. 3 for example. Such an arrangement provides an exit for guidewire 210 which is off-axis from lumen 245. The angle or configuration of diverter 248 can be modified as desired. In an embodiment, one or more of guidewire 210, lumen 245, and diverter 248 may include a lubricious coating to facilitate smooth operation. In an embodiment, diverter 248 may be constructed of a material different from body portion 244, such that the material of diverter 248 is of a lower friction than that of body portion 244.

Guidewire 210 can be generally similar to conventional guidewires, with a number of improvements. Guidewire 210 includes a body, a first end and a second end. First end includes a tip configured to facilitate piercing through body tissues. Tip may include one or more bevels to increase sharpness and/or aid delivery through introducer needle 240 and through the skin of a patient. First end of guidewire 210 can also include a pre-formed loop portion. In an embodiment wherein guidewire 210 is constructed of a shape-memory alloy such as nitinol, loop portion is set as the default shape for first end of guidewire 210. When introduced through needle 240, first end of guidewire 210 will conform to the shape of needle 240 while in lumen 245. In another embodiment, guidewire 210 may be generally straight without pre-formed loop portions.

The length and diameter of guidewire 210 may be selected as desired. In one embodiment, guidewire 210 has a diameter of approximately 0.035 inches and a length of approximately 32.0 inches. Further, the radius of curvature of loop portion 220 may be sized and selected as desired. In embodiments, the radius of curvature of loop portion 220 may be approximately 0.5 inches, approximately 0.75 inches, or approximately 1.0 inch.

Referring now to a method of use, in an embodiment the patient may undergo a CT scan or MRI prior to implant to determine the location and depth of the target implant site. In an embodiment, ultrasound may be used to identify the target implant site before the procedure (e.g., carotid sinus) and also during the procedure if needed. In an embodiment, needle 240 may include depth markings to guide insertion into the patient. A small incision is made caudally (inferior) of the location of the carotid sinus. Under ultrasound guidance, needle 240 is inserted into the neck downward and toward the carotid sinus. As a non-limiting example, needle 240 may be inserted at an angle of about 45 degrees with respect to the skin in the region of the target implant location.

Once distal tip 246 is near the target implant site, guidewire 210 may be advanced through lumen 245, onto diverter 248 and out of exit port 247. The angle and orientation of guidewire 210 exiting port 247 will vary depending on the angle of needle 240, the configuration of diverter 248, and whether guidewire 210 includes any pre-formed shape configurations. But in general, parameters of the devices disclosed herein should be selected such that guidewire 210 can be advanced from exit port 247 at a suitable angle toward the underside of the skin of the patient, before the tip of the guidewire 210 is advanced out through the skin. In a non-limiting example, the angle of guidewire 210 exiting port 247 may be approximately 45 degrees.

Once guidewire 210 has been successfully positioned in the patient, the remainder of the implant procedure then proceeds according to the description in the '145 PCT publication, the disclosure of which is incorporated by reference herein in its entirety.

Embodiments of the present disclosure may provide advantages over current generation baroreflex activation devices, with respect to stimulation efficacy and implant procedure. For example, embodiments of the present disclosure illustrate improved electrode structures (baroreflex activation devices) and improved systems for delivery and implantation of the improved electrode structures.

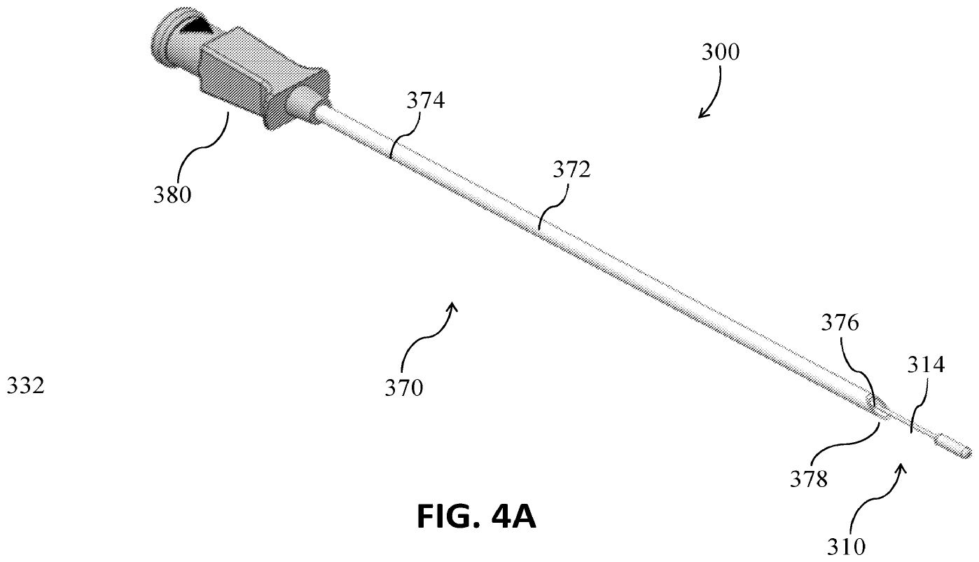

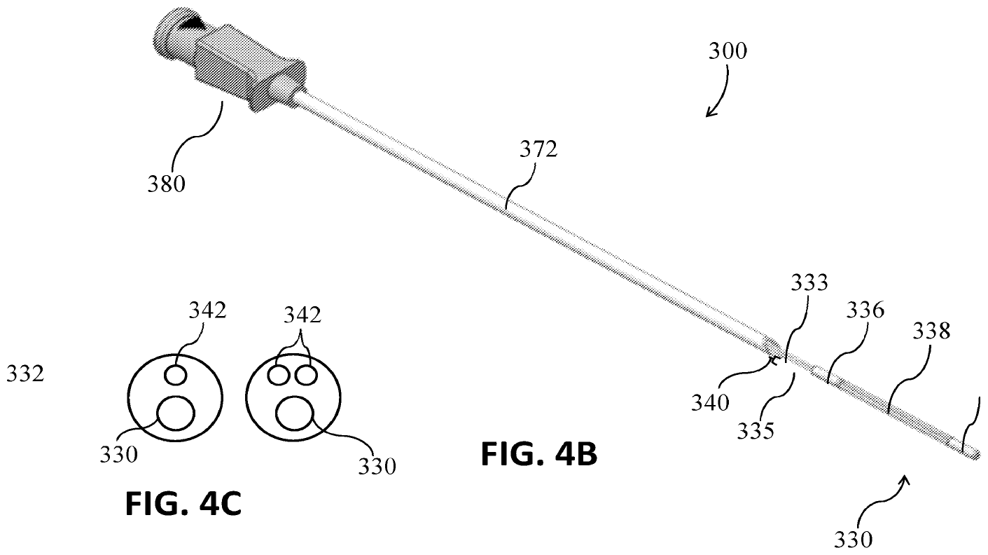

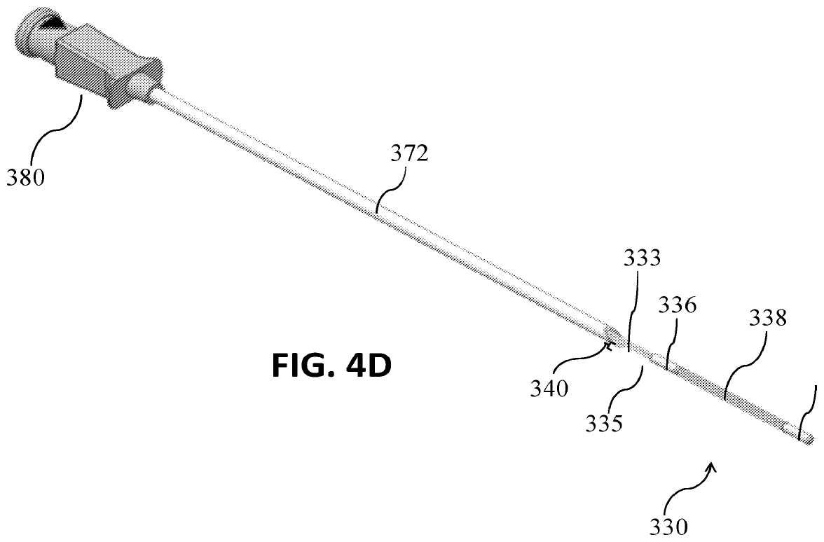

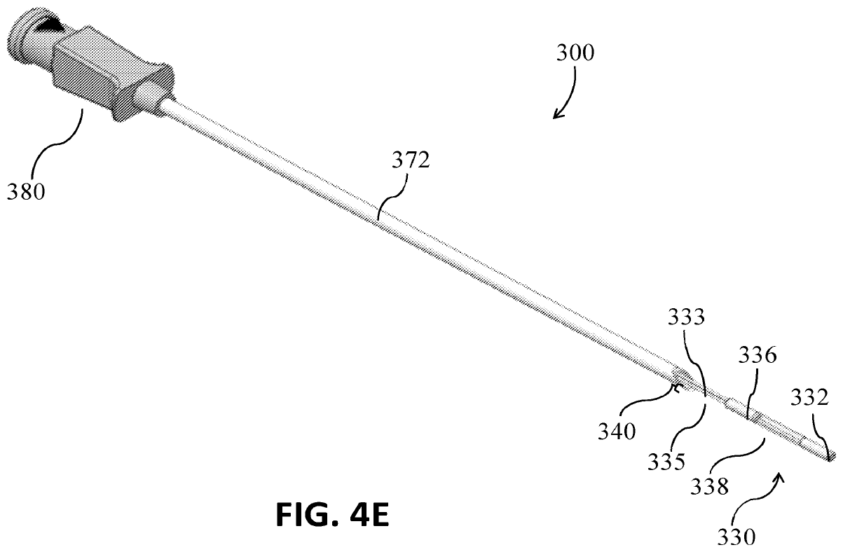

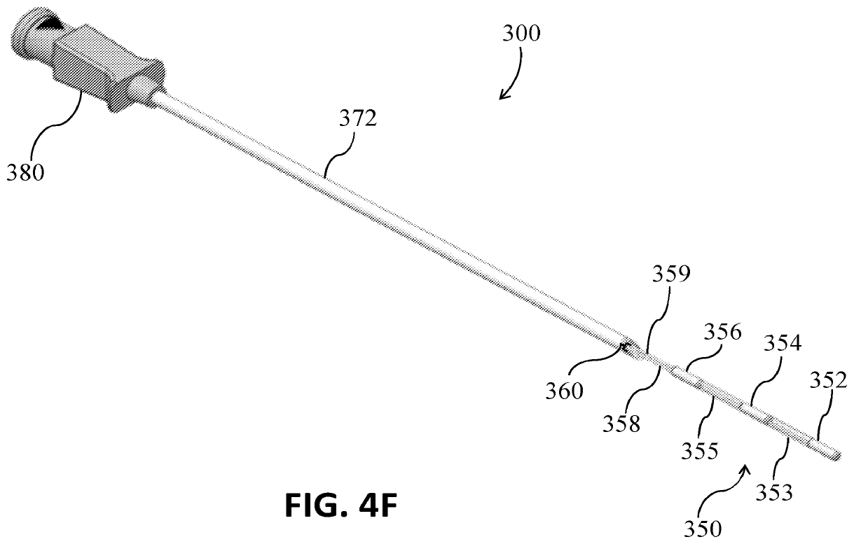

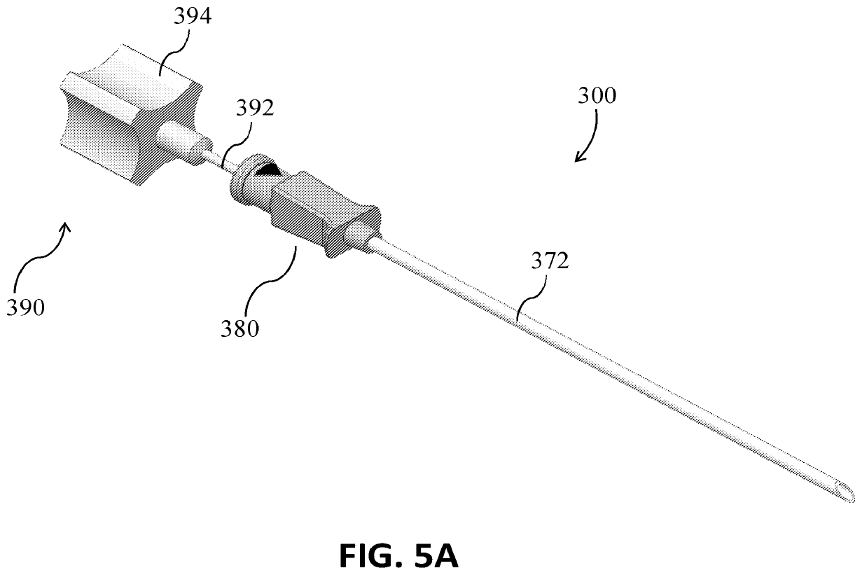

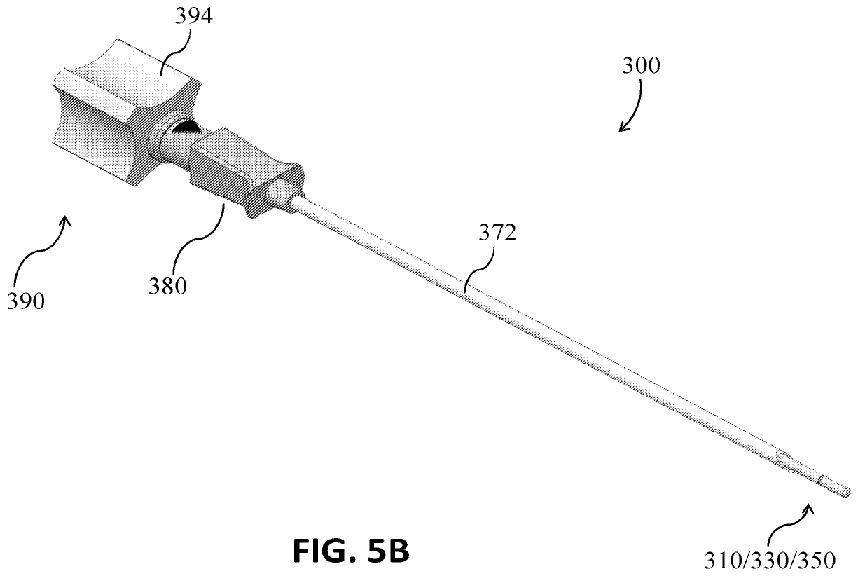

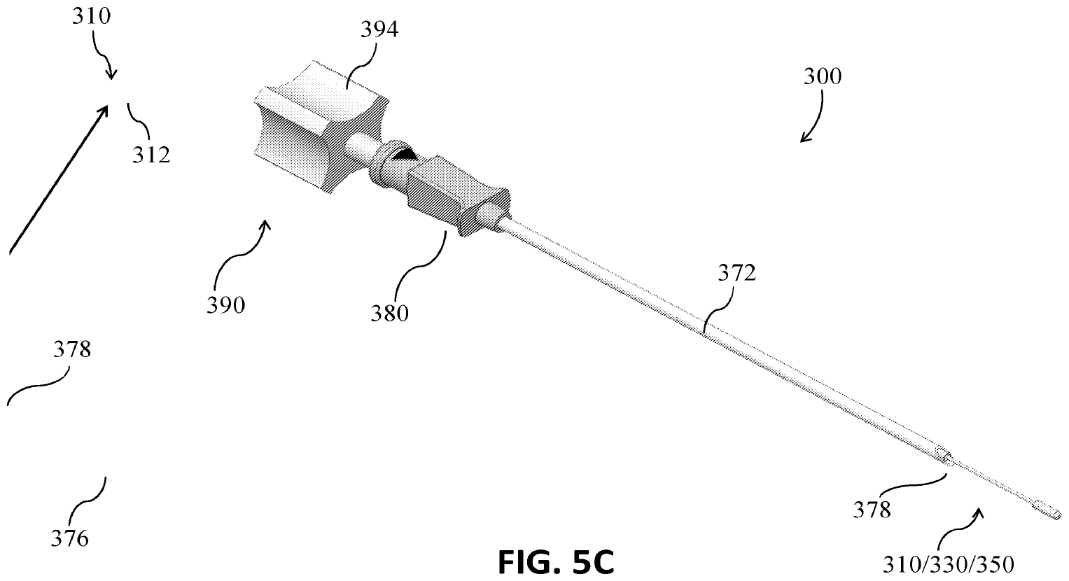

Referring now to FIGS. 4A-5C, a system 300 is depicted including baroreflex activation device 310/330/350, needle 370, and a plunger 390. As illustrated, a baroreflex activation device may be monopolar (reference numeral 310 in FIG. 4E), bipolar (reference numeral 320 in FIGS. 4A, 4B, 4D), or multipolar (reference numeral 330 in FIG. 4C) according to embodiments of the disclosure.

Baroreflex activation device 310 may include an electrode 312 coupled to a cable or conductor 314 configured to deliver a therapy signal from an implantable pulse generator to electrode 312. In embodiments, electrode 312 may be sized and shaped for introduction through the needle 370, and may take the form of a cylinder, a half-cylinder, or other suitable configuration. Electrode 312 may include masking on portions thereof, to tailor the electrical characteristics of electrode 312 as well as the baroreflex activation system 300 generally (for example, but not limited to, energy density and impedance tuning). Electrode 312 may be constructed from platinum and iridium, though other suitable materials known to one of ordinary skill are contemplated by this disclosure. Cable or conductor 314 may include appropriate insulation, and may be considered a lead body.

In embodiments, a shape element 342 may be included as part of monopolar baroreflex activation device 310, such as proximate conductor 314. In embodiments, the shape element 342 may comprise a stiffening member to provide structure to a portion of baroreflex activation device 310, such as to facilitate manipulation during an implant procedure. In an embodiment, the shape element 342 may comprise a biasing member such as a shape memory material (e.g., nitinol) to provide a predetermined shape to baroreflex activation device 310. In this manner, the shape element 342 can be manipulated to fit around a carotid artery during implantation of the baroreflex activation device 310 using system 300. In an embodiment, the shape element 342 may be configured in an arcuate shape correlated to a target implant location on an outer surface of a blood vessel. The shape element 342 may be integrated into or included within a portion of baroreflex activation device 310, such as conductor 314.

In an embodiment, electrode 312 may comprise a cathode, while the baroreflex activation therapy system 300 may further include an anode. In an embodiment, the surface area of the cathode may be approximately four times greater than the surface area of the anode, though other appropriate surfaces areas more than or less than four times are contemplated by this disclosure. In an embodiment, electrode 312 may comprise an anode. In embodiments, the baroreflex activation therapy system 300 may further include appropriate switching circuitry and components so as to switch electrode 312 between cathode and anode, as desired.

Baroreflex activation device 330 (depicted as bipolar in FIGS. 4A, 4B, and 4D) may include a first distal electrode 332 and a second proximal electrode 336, operably couplable via a link 338. A first conductor (or cable) 333 can be couplable to first electrode 332, and a second conductor 335 can be couplable to second electrode 336. Second electrode 336 may include a lumen or passageway therein to allow first conductor 333 to pass therethrough. First and second conductors 333, 335 may be bundled or otherwise grouped to form a lead body 330, with appropriate external insulation and/or internal lumens (e.g., introduction of stylets or other implant tools) included as part of lead body 340 as desired.

Each of electrodes 332, 336 may be sized and shaped for introduction through the needle 370, and may take the form of a cylinder, a half-cylinder, or other suitable configuration. Each of electrodes 332, 336 may be configured to include masking on portions thereof, to tailor the electrical characteristics of each electrode as well as the baroreflex activation system 300 generally (for example, but not limited to, energy density and impedance tuning). Each of electrodes 332, 336 may be constructed from platinum and iridium, though other materials known to one of ordinary skill in the art are contemplated by this disclosure.

In an embodiment, baroreflex activation device 330 may include one or more internal shape elements 342, either integrally constructed or selectively removable to an internal surface therein. In embodiments, shape element 342 may comprise a stiffening member to provide structure to a portion of baroreflex activation device 330, such as to facilitate manipulation during an implant procedure. In an embodiment, shape element 342 may comprise a biasing member such as a shape memory material (e.g., nitinol) to provide a predetermined shape to baroreflex activation device 330. In an embodiment, the shape element 342 may be configured into an arcuate shape correlated to a target implant location on an outer surface of a blood vessel. As depicted in FIG. 4C, for example, embodiments of baroreflex activation devices described herein may be configured to include one shape element 342 (left of FIG.) or two shape elements 342 (right of FIG.) so as to allow lesser or greater amounts of biasing.

Shape element 342 can be configured to create a semicircular orientation of baroreflex activation device 330 subsequent to delivery through the needle 370. In such an arrangement, baroreflex activation device 330 can be implanted on a target location (such as around a portion of a blood vessel containing baroreceptors) such that first electrode 332 and second electrode 336 can be arranged partially circumferentially around the target implant location. As implanted, the arrangement of first and second electrodes 332, 336 will advantageously reduce far field stimulation effects during delivery of baroreflex activation therapy, as well as improve capture of the target physiological structure (e.g., baroreceptors and/or associated nerves or nerve structures of a patient). In embodiments, shape element 342 may also be configured to slightly compress the target implant location (e.g., pinch) to aid with fixation of baroreflex activation device 330.

In an embodiment, first electrode 332 may comprise a cathode, while second electrode 336 comprises an anode. In an embodiment, first electrode 332 may comprise an anode, while second electrode 336 comprises a cathode. In an embodiment, the baroreflex activation therapy system 300 may further include a separate or additional anode. In embodiments, the baroreflex activation therapy system 300 may further include appropriate switching circuitry and components so as to switch each of electrodes 332, 336 between cathode and anode, as desired. In an embodiment, the surface area of the cathode may be approximately four times greater than the surface area of the anode, though other suitable surface areas more than or less than four times are contemplated by this disclosure. It will be understood that baroreflex activation device 330, with or without one or more shape elements 342, can be included or substituted for baroreflex activation device 310 in system 300.

Baroreflex activation device 350 (depicted as multipolar or split bipolar in FIG. 4E) may include a first distal electrode 352, a second middle electrode 354, and a third proximal electrode 356. First and second electrodes 352, 354 may be operably connected via a first link 353, while second and third electrodes 354, 356 may be operably connected via a second link 355. A first conductor 358 can be couplable to first electrode 352, and a second conductor 359 can be couplable to second electrode 354 and third electrode 356. In an embodiment, each of second electrode 354 and third electrode 356 may include separate, individual conductors rather than sharing second conductor 359.

Each of second and third electrodes 354, 356 may include one or more lumens or passageways therein to allow conductors to pass therethrough. First and second conductors 352, 354 may be bundled or otherwise grouped to form a lead body 430, with appropriate external insulation and/or internal lumens (e.g., introduction of stylets or other implant tools) included as part of lead body 360 as desired.

Each of electrodes 352, 354, 356 may be sized and shaped for introduction through the needle 370, and may take the form of a cylinder, a half-cylinder, or other suitable configuration. Each of electrodes 352, 354, 356 may be configured to include masking on portions thereof, to tailor the electrical characteristics of each electrode as well as the baroreflex activation system 300 generally (for example, but not limited to, energy density and impedance tuning). Each of electrodes 352, 354, 356 may be constructed from platinum and iridium, though other suitable materials known to one of ordinary skill in the art are contemplated by this disclosure.

By way of example and in no way limiting, in embodiments of the disclosure, cylindrical or half-cylinder electrodes described herein may have a length of approximately 0.150 inches and a diameter of approximately 0.035 inches. Other appropriate electrode lengths and diameters suitable for use with the baroreflex activation devices described herein are also contemplated by this disclosure. Accordingly, other suitable electrode form factors known to one of ordinary skill in the art are contemplated for use any of the baroreflex activation devices described herein.

In an embodiment, baroreflex activation device 350 may include one or more internal shape elements (similar or identical to shape elements 342), either integrally constructed or selectively removable. In embodiments, the shape element may comprise a stiffening member to provide structure to a portion of baroreflex activation device 350, such as to facilitate manipulation during an implant procedure. In an embodiment, the shape element may comprise a biasing member such as a shape memory material (e.g., nitinol) to provide a predetermined shape to baroreflex activation device 350. In an embodiment, the shape element may be configured into an arcuate shape correlated to a target implant location on an outer surface of a blood vessel. In an embodiment, the shape element may be configured such that baroreflex activation device 350 may be implanted similarly to the arrangement depicted in FIG. 4C. In embodiments, the shape element may also be configured to slightly compress the target implant location (e.g., pinch) to aid with fixation of baroreflex activation device 350.

In an embodiment, first electrode 352 may comprise a cathode, while second and third electrodes 354, 356 comprise an anode. Second and third electrodes 354, 356 may be configured as a common anode, or in another embodiment, configured as separate, individually selectable anodes so as to steer the stimulation field as desired. In embodiments, any of electrodes 352, 354, 356 may be configured as either cathode or anode, as desired. In embodiments, the baroreflex activation therapy system 300 may include appropriate switching circuitry and components so as to switch each of electrodes 352, 354, 356 between cathode and anode, as desired. In an embodiment, the surface area of the cathode may be approximately four times greater than the surface area of the anode, though other suitable surface areas more than or less than four times are contemplated by this disclosure. It will be understood that baroreflex activation device 350 can be included or substituted for baroreflex activation devices 310, 330 in system 300.

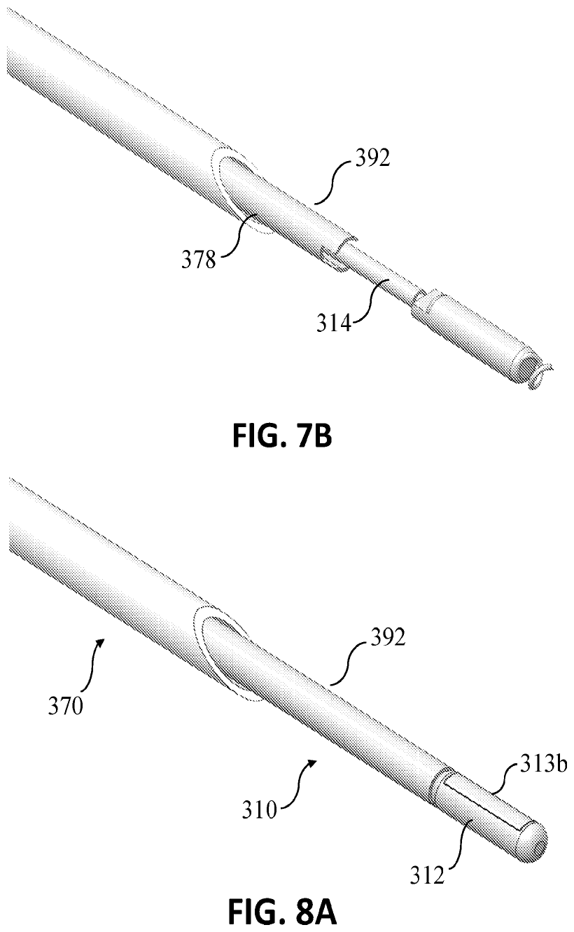

Embodiments of baroreflex activation devices 310/330/350 described herein may include one or more passive or active fixation elements as will be described later on in reference to FIGS. 7A-9C. Furthermore, embodiments of baroreflex activation devices 310/330/350 described herein having shape elements made from conductive materials (e.g., nitinol) may also include appropriate insulation or shielding as needed.

Needle 370 generally includes a body 372, internal lumen 374, distal tip 376 having a distal aperture 378, and a proximal end 380. Needle 370 may be angiographic or echogenic and may include a plurality of measurement markings along body 372, so as to assist during introduction of needle 370 into a patient. In one embodiment, needle 370 may be about 18 gauge, though other appropriate gauges greater than or less than 18 gauge are contemplated by this disclosure.

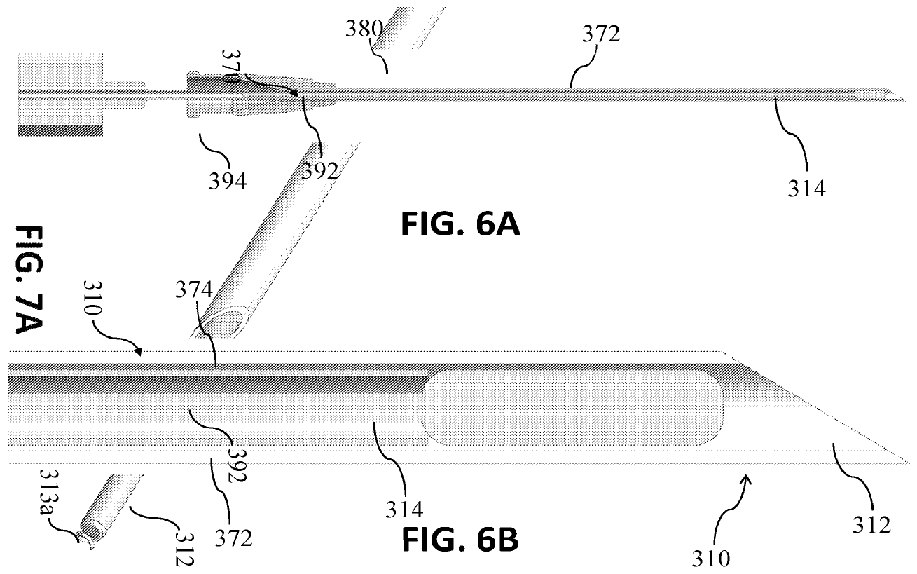

Referring specifically to FIGS. 5A-5C, plunger 390 generally includes a lumen 392 and a proximal handle 394. Plunger 390 is configured to facilitate delivery of a baroreflex activation device 310/330/350 from needle 370 into a patient, such that plunger 390 can be introduced within internal lumen 374 of needle 370 and abut at least a portion of baroreflex activation device 310/330/350 with needle 370, as depicted particularly in FIG. 6A.

As illustrated in FIG. 5A, plunger 390 may include an inactivated state in which proximal handle 394 has not been forced toward proximal end 380 of needle 370, such that lumen 392 is not fully enclosed within needle 370. As illustrated in FIG. 5B, once forced toward proximal end 380, a surface of plunger 390 is made coincident or substantially coincident with an opposing surface of proximal end 380 due to the change in position of the plunger 390. At this stage, lumen 392 is forced entirely within needle 370 such that baroreflex activation device 310/330/350 is forced partially out of needle 370.

As illustrated in FIG. 5C, baroreflex activation device 310/330/350 is forced further out of distal aperture 378 of needle 370 due to the activation of the plunger 390. Accordingly, delivery of the baroreflex activation device 310/330/350 into a patient is facilitated by the activation of the plunger 390 against the needle 370 (i.e., application of a force against the plunger 390 in a direction toward needle 370).

Referring now to FIGS. 6A and 6B, baroreflex activation device 310 is shown inside body 372 of needle 370 at a position where electrode 312 is not extended out of distal aperture 378 of distal tip 376. Cable or conductor 314 extends from electrode 312 through a substantial section of body 372 to an implantable pulse generator (not depicted) which can be configured to send therapy signals to the electrode 312 using the cable or conductor 314. A cross-sectional view of proximal end 380 of needle 370 is shown in FIG. 6A, with the cable or conductor 314 extending therethrough along with a portion of lumen 392 of plunger 390. Plunger 390 is shown in an unextended position which creates a distance between proximal handle 394 and a distal edge of proximal end 380. Although baroreflex activation device 310 is shown in the embodiments of FIGS. 6A and 6B, it should be understood that baroreflex activation devices 330/350 can be included or substituted therein and made to assume the position and configuration of baroreflex activation device 310 as presently depicted.

Referring now to FIGS. 7A and 7B, baroreflex activation device 310 of system 300 may include at least one anchoring feature in the form of a screw 313a extending from a surface of electrode 312. The anchoring feature may be described as a passive or active fixation element configured to connect the baroreflex activation device 310 to a carotid artery when implanted into a patient. For example, during implantation of the baroreflex activation device 310, electrode 312 can be positioned against a portion of the carotid artery such that screw 313a is inserted into the carotid which fixes the baroreflex activation device 310 in place at the implant site. After fixing the position of baroreflex activation device 310, needle 370 can be retracted in a direction opposite the insertion direction, as illustrated by FIG. 7B, to remove the needle 370 from the patient while retaining the fixated baroreflex activation device 310 at the implant site.

In embodiments, screw 313a may be attached to a distal tip of electrode 312 or to a peripheral surface of electrode 312, or to both the distal tip and the peripheral surface where more than one screw 313a is included with electrode 312. Screw 313a may be fixedly couplable to electrode 312 (e.g., integrated directly into electrode 312 during manufacturing) or removably couplable to electrode 312 such that screw 313a can be removed and replaced as needed. It should be understood that screw 313a can assume a variety of forms and geometries suitable for use with baroreflex activation device 310, including having many different lengths, widths, and diameters, such that screw 313a is depicted only by way of example in FIGS. 7A and 7B and should not be limited to the form and geometry shown in these illustrative depictions.

Although baroreflex activation device 310 is shown in the embodiments of FIGS. 7A and 7B, it should be understood that baroreflex activation devices 330/350 can be included or substituted therein and made to assume the position and configuration of baroreflex activation device 310 as presently depicted. For example, screw 313a can be connected to first distal electrode 332 of baroreflex activation device 330, or first electrode 352 of baroreflex activation device 350, in a manner identical to that as depicted in FIGS. 7A and 7B.

In embodiments, baroreflex activation devices 330/350 may include more than one screw 313a attached to, respectively, the first distal electrode 332 and the first electrode 352. In embodiments, baroreflex activation device 330 may include one or more screws 313a on second proximal electrode 336 in a manner similar to that of first distal electrode 332. In embodiments, baroreflex activation device 350 may include one or more screws 313a on one or both of second and third electrodes 354, 356 in a manner similar to that of first electrode 352.

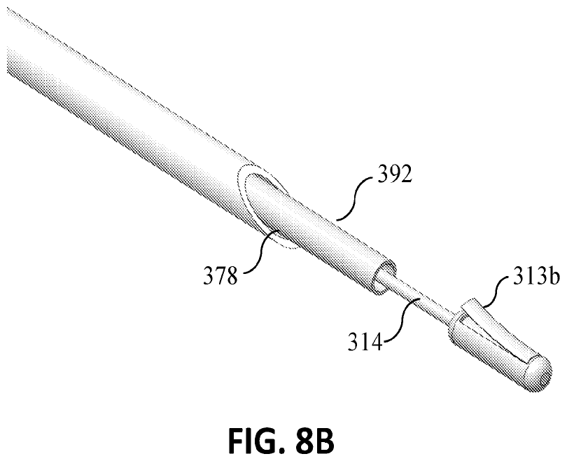

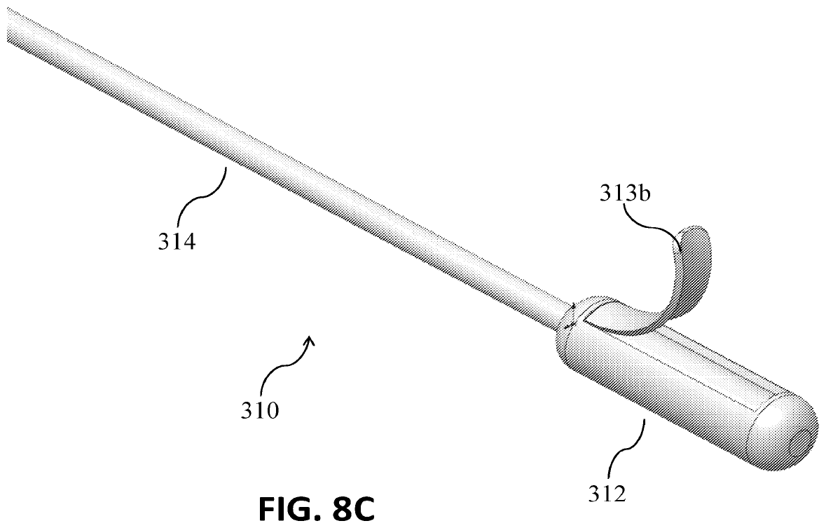

Referring now to FIGS. 8A-8C, baroreflex activation device 310 of system 300 may include at least one anchoring feature in the form of a tab 313b extending from a surface of electrode 312. As with screw 313a, the anchoring feature may be described as a passive or active fixation element configured to connect the baroreflex activation device 310 to a carotid artery when implanted into a patient. Accordingly, tab 313b is designed to achieve the same objective as screw 313a of securely fixing the baroreflex activation device 310 to the carotid artery at the implant site after needle 370 is removed from the patient.

For example, during implantation of the baroreflex activation device 310, electrode 312 can be positioned against a portion of the carotid artery such that tab 313b is placed in contact and inserted into the carotid which fixes the baroreflex activation device 310 in place at the implant site. After fixing the position of baroreflex activation device 310, needle 370 can be retracted in a direction opposite the insertion direction, as illustrated by FIG. 8B. This removes needle 370 from the patient while retaining the baroreflex activation device 310 at the implant site with tab 313b inserted at least partially into the carotid artery of the patient.

Generally, tab 313b can be designed to have a rectangular geometry with or without rounded edges as shown in FIGS. 8A-8C. Other suitable geometries, including circular, triangular, cylindrical, etc., are also contemplated by the present disclosure. In operation, baroreflex activation device 310 with one or more tabs 313b can be inserted into needle 370 in preparation for being implanted into a patient. When baroreflex activation device 310 extends out of needle 370, the one or more tabs 313b are generally positioned flat against the surface of electrode 312 such that the one or more tabs 313b are attached to the electrode 312 on both ends, as illustrated by FIG. 8A.

After positioning the electrode 312 against a portion of the carotid artery, a portion of each one of the one or more tabs 313b can be translated outward from electrode 312 so as to form an arcuate shape when viewed from the side (as illustrated by FIGS. 8A and 8B). In this manner, each tab 313b becomes unattached from electrode 312 on one end while retaining attachment to the electrode 312 on the other end. Because electrode 312 is positioned against the carotid artery such that the one or more tabs 313b are in direct contact with the carotid, the portion(s) of the one or more tabs 313b extending outward may become implanted within the carotid which fixes the baroreflex activation device 310 in place at the implant site.

In embodiments, tab 313b may be attached to a distal tip of electrode 312 or to a peripheral surface of electrode 312, or to both the distal tip and the peripheral surface where more than one tab 313b is included with electrode 312. Tab 313b may be fixedly couplable to electrode 312 (e.g., integrated directly into electrode 312 during manufacturing) or removably couplable to electrode 312 such that tab 313b can be removed and replaced as needed. It should be understood that tab 313b can assume a variety of forms and geometries suitable for use with baroreflex activation device 310, including having many different lengths, widths, and diameters, such that tab 313b is depicted only by way of example in FIGS. 8A-8C and should not be limited to the form and geometry shown in these illustrative depictions.

Although baroreflex activation device 310 is shown in the embodiments of FIGS. 8A-8C, it should be understood that baroreflex activation devices 330/350 can be included or substituted therein and made to assume the position and configuration of baroreflex activation device 310 as presently depicted. For example, tab 313b can be connected to first distal electrode 332 of baroreflex activation device 330, or first electrode 352 of baroreflex activation device 350, in a manner identical to that as depicted in FIGS. 8A-8C. In embodiments, baroreflex activation devices 330/350 may include more than one tab 313b attached to, respectively, the first distal electrode 332 and the first electrode 352. In embodiments, baroreflex activation device 330 may include one or more tabs 313b on second proximal electrode 336 in a manner similar to first distal electrode 332. In embodiments, baroreflex activation device 350 may include one or more tabs 313b on one or both of second and third electrodes 354, 356 in a manner similar to that of first electrode 352.

Referring now to a method of implant, in an embodiment the patient may undergo a CT scan or MRI prior to implant to determine the location and depth of the target implant site. Needle 370 can be inserted into the patient toward the implant site, utilizing depth markings on the needle 370 to guide the insertion. Once needle 2370 is in the general vicinity of the implant site, in an embodiment a camera can be advanced down the needle to inspect the implant site. With needle 370 satisfactorily located, the baroreflex activation device 310/330/350 can be loaded into needle 370 as depicted, for example, in FIGS. 6A and 6B.

In embodiments, the distal electrode (332 or 352) can be at least partly advanced out of needle 370 prior to final deployment to allow a mapping procedure at the target implant site. Mapping in the context of baroreflex activation therapy systems, including system 300, generally involves moving the stimulating electrode to different locations around the target implant site, delivering one or more therapy signals, and determining one or more patient physiological responses to the therapy signals. Examples of mapping procedures are described in U.S. Pat. No. 6,850,801 to Kieval et al. and U.S. Pat. No. 9,345,877 to Pignato et al., the disclosures of which are incorporated by reference herein in their entireties.

To fully deploy the baroreflex activation device 310/330/350, it will be understood that in one approach, the baroreflex activation device 310/330/350 may be held in place, such as with plunger 390, and needle 370 can be retracted with respect to the baroreflex activation device 310/330/350. In another approach, needle 370 may be held in place, and the baroreflex activation device 310/330/350 can be advanced forward with respect to needle 370, such as by applying a force against plunger 390 in the direction of needle 370. It will also be understood that a combination of approaches may be used. By way of example, FIGS. 5A-5C depict a sequence of images of a baroreflex activation device 310/330/350 emerging from needle 370, according to embodiments of the disclosure. Similar illustrations of deploying the baroreflex activation device 310/330/350 are illustrated in FIGS. 7A and 7B, 8A and 8B, and 9A-9C which depict deployment of the baroreflex activation device 310/330/350 against a carotid sinus C.

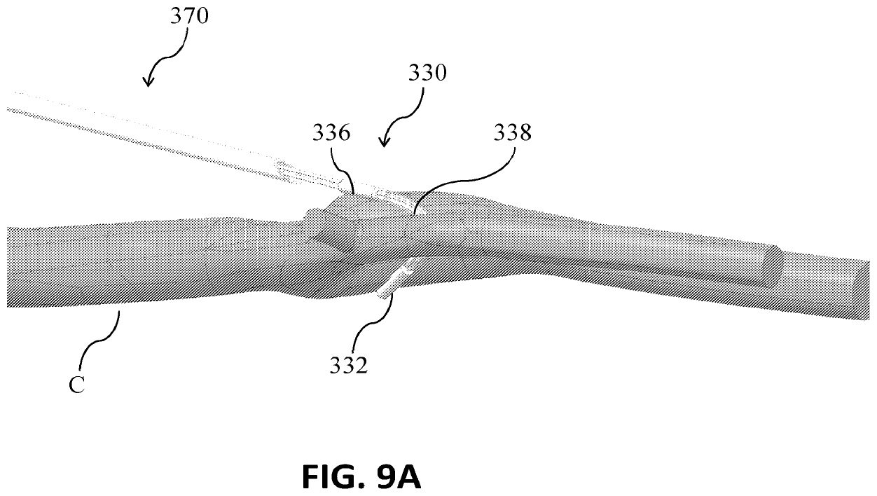

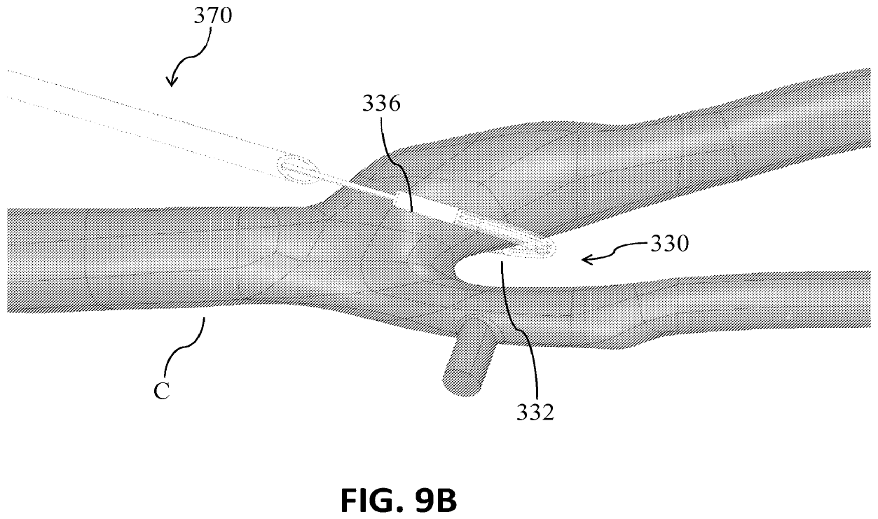

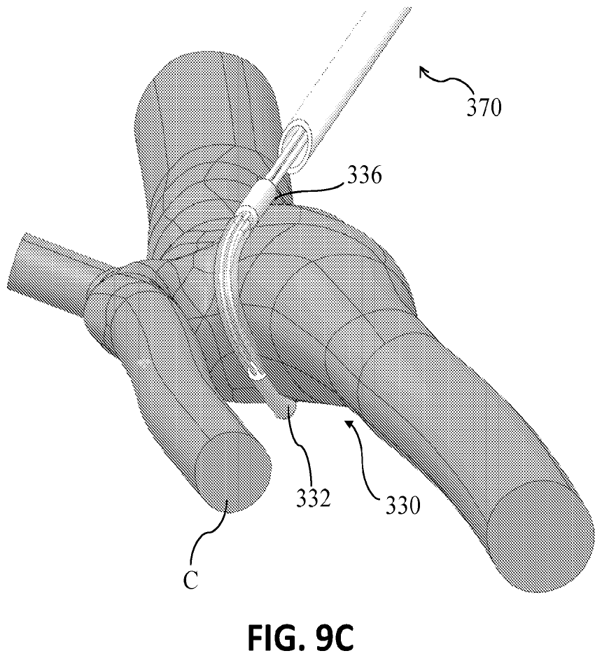

Referring now specifically to FIGS. 9A-9C, an example implant orientation of a baroreflex activation device 330 is depicted, according to embodiments of the present disclosure (bipolar, with first distal electrode 332 and second proximal electrode 336 arranged around the carotid sinus C). Baroreflex activation device 330 is constructed according to the embodiments of FIGS. 4B, 4D, and 4E, and is depicted schematically on a carotid sinus C of a patient with first distal electrode 332 on the “backside” of the carotid (or medial) C, with the second proximal electrode 336 on the “frontside” (or lateral) of the carotid C. A shape element within link 338 can create a biasing effect to curve baroreflex activation device 330 at least partially circumferentially around the carotid C (i.e., to give baroreflex activation device 330 an arcuate shape when viewed from the side).

To implant baroreflex activation device 330 in such a manner, it will be understood that introducer needle 370 may need to be advanced “past” the target and then subsequently deployed, allowing first distal electrode 332 enough space to cuff around the carotid artery C in an arcuate shape.

Although not depicted, it should be understood that baroreflex activation device 330 can be replaced with baroreflex activation devices 310/350 in FIGS. 9A-9C. Baroreflex activation devices 310/350 can be positioned similarly to baroreflex activation 330, taking into account the use of a single electrode 312 with baroreflex activation device 310 (e.g., the arcuate shape can be formed in a portion of cable or conductor 314) and three electrodes 352, 354, 356 with baroreflex activation device 350 (e.g., the arcuate shape can be formed in portions of first and second links 353, 355).

Moreover, it should be understood that one or more anchoring features, such as screw 313a or tab 313b, can be included with the baroreflex activation device 330 depicted in FIGS. 9A-9C. For example, first distal electrode 332 can include a tab 313b that extends from a peripheral surface of the electrode 332 into the “frontside” of the carotid sinus C, while second proximal electrode 336 can include a screw 313a extending from a peripheral surface of electrode 336 into the “backside” of the carotid sinus C. First distal electrode 332 and second proximal electrode 336 can include a combination of one or more screws 313a and tabs 313b to provide additional fixation points between baroreflex activation device 330 and the carotid C. This is in addition (or alternatively) to fixation that may be provided by the arcuate shape formed by the positioning of the first and second electrodes on the adjacent “backside” and “frontside”surfaces of the carotid C.

Various embodiments of systems, devices, and methods have been described herein. These embodiments are given only by way of example and are not intended to limit the scope of the disclosure. It should be appreciated, moreover, that the various features of the embodiments that have been described may be combined in various ways to produce numerous additional embodiments. Moreover, while various materials, n dimensions, shapes, configurations and locations, etc. have been described for use with disclosed embodiments, others besides those disclosed may be utilized without exceeding the scope of the disclosure.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1-5. (canceled)

6. A method for implanting a baroreflex activation device into a patient, comprising:
inserting a needle into the patient at an implant site near a carotid artery, the needle having a body and a lumen defined along the body, the needle inserted in a first direction toward the carotid artery;
loading the baroreflex activation device into a proximal end of the needle;
advancing the baroreflex activation device along the lumen until a distal electrode of the baroreflex activation device extends beyond a distal tip of the needle;
positioning the distal electrode of the baroreflex activation device against the carotid artery; and
retracting the needle in a second direction opposite the first direction until the needle is removed from the patient.

7. The method of claim 6, wherein the baroreflex activation device comprises a distal electrode and one or more proximal electrodes, the distal electrode being connected to the one or more proximal electrodes via a link housing one or more conductors.

8. The method of claim 7, wherein the link includes one or more shape elements made from a shape memory material, configured to bias the link into an arcuate shape upon deployment.

9. The method of claim 6, wherein the needle includes an exit port positioned along the body, configured to facilitate off-axis deployment of the baroreflex activation device.

10. The method of claim 9, wherein the exit port is positioned proximal to the distal

11. The method of claim 6, wherein the distal electrode includes an anchoring feature selected from the group consisting of a tab and a screw for fixation at or near the carotid artery.

12. The method of claim 6, wherein the baroreflex activation device is secured to the carotid artery using passive fixation elements embedded within the device.

13. The method of claim 6, wherein the baroreflex activation device comprises a monopolar, bipolar, or multipolar electrode configuration for baroreflex activation therapy.

14. The method of claim 6, further comprising using a plunger to advance the baroreflex activation device through the lumen of the needle.

15. The method of claim 14, wherein the plunger facilitates controlled movement of the baroreflex activation device from the proximal end of the needle to the distal tip.

16. The method of claim 14, further comprising rotating the needle or adjusting the plunger to control the orientation of the baroreflex activation device during deployment.

17. The method of claim 6, wherein the needle includes depth control markings to facilitate positioning within a baroreceptive tissue plane.

18. The method of claim 6, further comprising advancing a guidewire through the lumen, the guidewire being directed out of an exit port of the needle using a diverter.

19. The method of claim 18, wherein the diverter is configured as a ramp, wedge, slope, or curve to facilitate direction control of the guidewire or baroreflex activation device.

20. The method of claim 18, wherein the distal tip of the needle is closed, preventing direct passage of the guidewire through the end of the needle.

21. The method of claim 18, wherein the guidewire comprises a shape memory alloy configured to curve upon deployment.

22. The method of claim 6, wherein the baroreflex activation device is configured to deliver electrical stimulation to baroreceptors upon fixation at or near the carotid artery.

23. The method of claim 6, wherein the needle is inserted into the patient with positioning guided by imaging means to facilitate access to the carotid artery while maintaining depth control.

24. The method of claim 6, wherein the baroreflex activation device includes a wraparound, looped, or semi-rigid element configured to circumferentially engage the carotid artery.

25. The method of claim 6, wherein the baroreflex activation device is configured to adjust therapy signal parameters based on a measured sensor signal indicative of a patient's physiological state.
